(19) United States
(12) Patent Application Publication
(10) Pub. No.: US 2002/0035161 A1
(43) Pub. Date: Mar. 21, 2002

(54) O/W emulsions comprising micronized biologically active agents

(76) Inventors: Sandrine Segura, Biot (FR); Isabelle Preuilh, Cannet (FR)

Correspondence Address: Norman H. Stepno, Esquire, BURNS, DOANE, SWECKER & MATHIS, L.L.P, P.O. Box 1404, Alexandria, VA, 22313-1404, US

(21) Appl. No.: 09/881,686

(22) Filed: Jun. 18, 2001

Related U.S. Application Data
(63) Continuation of application No. PCT/FR99/03136, filed on Dec. 14, 1999.

(30) Foreign Application Priority Data
Dec. 18, 1998 (FR) 98/16050

(51) Int. Cl. A61K 7/00; A61K 47/32

(52) U.S. Cl. 514/772.6; 424/401

(57) ABSTRACT

Topically applicable cosmetic/pharmaceutical oil-in-water emulsions, well suited for treating or caring for the skin and/or superficial body growths therefrom, include a discontinuous fatty phase dispersed in a continuous aqueous phase and comprise an effective amount of at least one biologically active agent (A) and an effective amount of an emulsifying system (B) therefor, the at least one biologically active agent (A) being non-solubilized therein in micronized particulate state, at least 80%, numerically, of the micronized particles having diameters ranging from 1 to 10 μm and at least 50%, also numerically, having diameters of less than 5 μm.

### CROSS-REFERENCE TO PRIORITY/PCT APPLICATIONS

This application claims priority under 35 U.S.C. §119 of FR-98/16050, filed Dec. 18, 1998, and is a continuation of PCT/FR99/03136, filed Dec. 14, 1999 and designating the United States (published in the French language on Jun. 29, 2000 as WO 00/37027; the title and abstract were also published in English), both hereby expressly incorporated by reference.

### BACKGROUND OF THE INVENTION

1. Technical Field of the Invention

The present invention relates to novel cosmetic or pharmaceutical compositions comprising oil-in-water (O/W) emulsions containing a micronized biologically active agent and a suitable emulsifying system therefor, and to the topical application of such novel cosmetic/pharmaceutical compositions, to treat or care for the skin and/or the superficial body growths therefrom.

The compositions of the present invention are particularly well suited for promoting the penetration of the biologically active agent to the base of hair follicles.

This invention also relates to novel compositions for treating and/or preventing any affliction associated with an inflammation or infection of the tissues surrounding the hair follicles.

2. Description of the Prior Art

A wide variety of dermatological compositions comprising an active agent are known in the prior art for the treatment of acne, for example. For various reasons associated, in particular, with excess sebum and the tendency of acneic skin towards greasiness, these compositions are usually in the form of aqueous gels. While having a non-greasy feel and providing a sensation of freshness, aqueous gels present the drawback of producing a sensation of tautness of the skin, which is also uncomfortable, when they are applied very frequently.

The active principle(s) in the known cosmetic or pharmaceutical compositions is (are) generally in solubilized form. However, it has been determined that certain active principles are relatively insoluble at a pH of from 5 to 7, i.e., at a pH which is compatible with the skin and at a pH which is ideal for a highly tolerated composition. It is thus impossible to administer same in solubilized form at such a pH without incorporating additives therefor. It is thus necessary for the active principle to be in a thermodynamic state other than solubilization.

Furthermore, for reasons of efficacy or to avoid eliciting adverse side effects, it is occasionally preferable to administer the active principle selectively to target or site-specific zones.

This may be the case, for example, for the treatment of certain skin conditions and/or afflictions and/or afflictions of superficial body growths, during which it is preferable to render the active principle selectively available to the base of the hair follicles, such as for the treatment of dermatological conditions/afflictions associated with an inflammation or infection of the tissues surrounding the hair follicles. Among these conditions/afflictions, particularly exemplary are acne and folliculitis.

### SUMMARY OF THE INVENTION

Accordingly, a major object of the present invention is the provision of novel topically applicable cosmetic/pharmaceutical compositions which are quite comfortable upon administration, which promote delivery of a biologically active principle to a site at which it is intended to elicit its desired bioaffecting response, without modifying the activity thereof or its compatibility with the skin, scalp and/or hair, and which otherwise avoid or conspicuously ameliorate the above disadvantages and drawbacks to date characterizing the state of this art.

Briefly, it has now unexpectedly and surprisingly been determined that formulating a biologically active compound in micronized and non-solubilized form, as a dispersion in an emulsion of oil-in-water type comprising a suitable emulsifying system, provides pharmaceutical/cosmetic compositions which do not exhibit the drawbacks of the counterpart compositions of the prior art.

Thus, the present invention features cosmetic/pharmaceutical compositions of oil-in-water emulsion type including a fatty phase dispersed in an aqueous phase, which comprise:

(A) at least one non-solubilized, micronized, biologically active compound in particle form, in which at least 80% by number of the particles and preferably at least 90% by number of the particles have a diameter ranging from 1 to 10 μm and at least 50% by number of the particles have a diameter of less than 5 μm, and

(B) a suitable emulsifying system therefor.

### DETAILED DESCRIPTION OF BEST MODE AND SPECIFIC/PREFERRED EMBODIMENTS OF THE INVENTION

More particularly according to the present invention, the subject emulsions present the advantage of being compatible with the skin and of feeling comfortable when topically applied, without being greasy or sticky, while at the same time promoting the selective penetration of the biologically active compound into the hair follicles, thus increasing its efficacy and reducing the adverse side effects thereof.

Another advantage of the emulsions of the invention is that it is not necessary to encapsulate the biologically active compound or species, this technique being employed in the prior art to effect targeting of the hair follicles. The absence of encapsulation simplifies the process for manufacturing formulations of the active compound and thus reduces costs.

According to the invention, advantageously, in the subject compositions, the emulsifying system comprises at least one copolymer prepared from a major fraction of monoolefinically unsaturated C₃-C₆ carboxylic acid monomer or anhydride thereof and a minor fraction of acrylic acid ester monomer containing a fatty chain.

The emulsifying copolymers in accordance with the present invention are prepared by polymerizing a predominant amount of monoolefinically unsaturated carboxylic acid monomer or anhydride thereof, with a smaller amount of acrylic acid ester monomer containing a fatty chain. The amount of carboxylic acid monomer or anhydride thereof preferably ranges from 80% to 98% by weight and more particularly from 90% to 98% by weight, while the acrylic acid ester containing a fatty chain is advantageously present in amounts of from 2% to 20% by weight and more particularly from 1% to 10% by weight; the percentages being calculated relative to the total weight of the two monomers.

The preferred carboxylic acid monomers are selected from among those having the following structural formula:

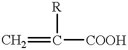

in which R is hydrogen, halogen, hydroxyl, a lactone group, a lactam group, a cyanogen (—C═N) group, a monovalent alkyl radical, an aryl radical, an alkylaryl radical, an aralkyl radical or a cycloaliphatic radical.

The carboxylic acid monomers which are particularly preferred are acrylic acid and methacrylic acid, or mixtures thereof.

The acrylic acid ester monomers containing a fatty chain are preferably selected from among those having the structural formula:

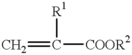

in which R¹ is hydrogen, methyl or ethyl and R² is a C₈-C₃₀ alkyl radical.

The ester monomers which are particularly preferred are those in which R¹ is hydrogen or methyl and R² is a C₁₀-C₂₂ alkyl radical.

The emulsifying copolymers of the invention are described in EP-A-0,268,164 and are prepared according to the methodology set forth therein.

The acrylate/C₁₀-C₃₀-alkylacrylate copolymer such as the product marketed under the trademark Pemulen TR 1 or the product marketed under the trademark Carbopol 1342 by Goodrich, or mixtures thereof, are more particularly preferred.

The emulsions of the invention can also contain other surfactant emulsifiers. Exemplary of these compounds are glyceryl (and) PEG-100 stearate marketed under the trademark Arlacel 165 by ICI or under the trademark Simulsol 165 by SEPPIC, polyoxyethylenated fatty acid esters such as Arlatone 983 marketed by ICI, or polyoxyethylenated stearyl alcohol (2) marketed under the trademark Brij72 combined with polyethylenated stearyl alcohol (21) marketed under the trademark Brij721 by ICI.

The emulsions of the invention can also contain co-surfactants. Among these compounds which are exemplary thereof are sorbitan esters such as sorbitan oleate marketed under the trademark Arlacel 80 by ICI or marketed under the trademark Crill 4 by Croda, sorbitan sesquioleate marketed under the trademark Arlacel 83 by ICI or marketed under the trademark Montane 83 by SEPPIC, or sorbitan isostearate; fatty alkyl ethers with a high HLB value, i.e., an HLB value of greater than or equal to 7, such as ceteareth-20 or ceteareth-12, or fatty alkyl ethers with a low HLB value, i.e., an HLB value of less than 7, such as steareth-2.

The compositions according to the present invention advantageously comprise up to 15% by weight of suitable emulsifying system, preferably 0.05% to 8% by weight and more preferably from 0.1% to 2% by weight relative to the total weight thereof.

In the emulsifying system, the amount of copolymer can range, for example, from 0.01% to 3% by weight relative to the total weight of the composition. When the emulsifying system is an acrylate/C₁₀-C₃₀-alkylacrylate copolymer, the amount of copolymer preferably ranges from 0.05% to 2% and more preferably from 0.1% to 0.5% by weight relative to the total weight of the composition.

Any biologically active agent which is insoluble or difficult to dissolve in water or in a hydrophilic medium under pH conditions which are compatible with the skin, i.e., a pH of from 5 to 7, and which can be micronized, is well suited for formulation into the emulsions of the present invention.

By the expression “biologically active compound” is intended any compound capable of modifying or modulating the function of at least one given biological system, mechanism, or cascade.

Exemplary such biologically active agents include those species or agents which modulate skin differentiation and/or proliferation and/or pigmentation, antibacterial agents, antiparasitic agents, antifungal agents, antibiotics, steroidal anti-inflammatory agents, non-steroidal anti-inflammatory agents, anaesthetics, anti-pruriginous agents, antiviral agents, keratolytic agents, free-radical scavengers, antiseborrhoeic agents, antidandruff agents, anti-acne agents, antimetabolites, agents for combating hair loss and for promoting hair growth or vice versa, and antiseptics, or mixtures thereof.

Representative active agents for modulating differentiation and/or proliferation, for example, are the retinoids. Exemplary such retinoids include adapalene, all-trans-retinoic acid and acidic retinoids containing at least one carboxylic function. And exemplary acidic retinoids include 6-[7-(1-adamantyl)-6-methoxyethoxymethoxy-2-naphthyl]nicotinic acid, 6-[3-(1-adamantyl-4-hydroxyphenyl]-2-naphthoic acid, 6-(3,5,5,8,8-pentamethyl-5,6,7,8-tetrahydro-2-naphthylthio)nicotinic acid, 3-(3,5,5,8,8-pentamethyl-5,6,7,8-tetrahydro-2-naphthyl)phenylacrylic acid and 2-hydroxy-4-[7-(1-adamantyl)-6-methoxyethoxymethoxy-2-naphthyl]benzoic acid, or mixtures thereof.

Exemplary antibiotics include the fluoroquinolones, rifamycin, josamycin, sulfadiazine, virginiamycin and fusidic acid, or mixtures thereof. Fluoroquinolones and more particularly nadifloxacin are the preferred.

Among the antibacterial agents which are representative, for example, is benzoyl peroxide.

Among the antidandruff agents which are representative, for example, is piroctone olamine.

Among the keratolytic agents which are representative, for example, is salicylic acid.

Among the free-radical scavengers which are representative, for example, is vitamin E.

Among the antiparasitic agents which are representative, for example, is crotamiton.

Representative of the antiviral agents is Vidarabine.

Representative of the antifungal agents are griseofulvin, compounds belonging to the imidazole class such as econazole, ketoconazole or miconazole, polyene compounds such as amphotericin B, compounds of the allylamine family such as terbinafine, or, alternatively, piroctone olamine.

And representative of steroidal anti-inflammatory agents are clobetasone butyrate, hydrocortisone, fluocinolone acetonide and betamethasone.

The compositions of the invention are particularly suitable for treating the following conditions/afflictions and/or disease states:

(1) dermatological conditions/afflictions associated with a keratinization disorder relating to differentiation and proliferation, in particular for treating common acne, comedones, polymorphonuclear leukocytes, rosacea, nodulocystic acne, acne conglobata, senile acne and secondary acne such as solar, medication-related or occupational acne;

(2) other types of keratinization disorders, in particular ichtyosis, ichtyosiform states, Darier's disease, palmoplantar keratoderma, leukoplasia and leukoplasiform states, and cutaneous or mucous (buccal) lichen;

(3) other dermatological conditions/afflictions associated with a keratinization disorder including an inflammatory and/or immunoallergenic component and, in particular, all forms of psoriasis, whether cutaneous, mucous or ungual psoriasis, and even psoriatic rheumatism, or cutaneous atopy, such as eczema or respiratory atopy or gingival hypertrophy; the subject compositions are also useful for treating certain inflammatory conditions which manifest no keratinization disorder, such as rosacea;

(4) all dermal or epidermal proliferations, whether benign or malignant and whether of viral or non-viral origin, such as common warts, flat warts and verruciform epidermodysplasia, it also being possible for the oral or florid papillomatoses and proliferations to be induced by ultraviolet radiation, in particular in the event of basocellular and spinocellular epithelioma;

(5) other dermatological disorders such as bullosis and collagen diseases;

(6) for repairing or combating aging of the skin, whether light-induced or chronological aging, or for reducing pigmentations and actinic keratosis, or any pathologies associated with chronological or actinic aging;

(7) for preventing or curing the stigmata of epidermal and/or dermal atrophy induced by local or systemic corticosteroids, or any other form of cutaneous atrophy;

(8) for the preventive or curative treatment of cicatrization disorders, for preventing or repairing stretch marks or for promoting cicatrization;

(9) for combating disorders of sebaceous functioning, such as acneic hyperseborrhoea or simple seborrhoea;

(10) for the preventive or curative treatment of cancerous or precancerous states;

(11) for the treatment of inflammatory conditions such as arthritis;

(12) for the treatment of any skin complaint of viral origin;

(13) for the preventive or curative treatment of alopecia;

(14) for the treatment of dermatological conditions including an immunological component;

(15) for the treatment of skin disorders due to exposure to UV radiation;

(16) for the treatment of dermatological conditions associated with inflammation or infection of the tissues surrounding the hair follicles, in particular due to colonization or microbial infection, in particular impetigo, seborrhoeic dermatitis, folliculitis or sycosis barbae, or a treatment involving any other bacterial or fungal agent;

(17) for cosmetic treatments to accelerate or promote hair loss.

The compositions of the invention are particularly suitable for the preventive or curative treatment of acne.

For the treatment of acne, the biologically active compounds are preferably selected from among the antibiotics, antibacterial agents, antifungal agents, antiparasitic agents and retinoids, or mixtures thereof.

The amounts of the biologically active compound, agent, or species, in the compositions of the invention will of course depend on the particular biologically active compound concerned and on the quality of the treatment desired.

To provide an order of magnitude, the compositions according to the invention advantageously comprise from 0.0001% to 20% by weight relative to the total weight of the composition of the biologically active compound, and preferably from 0.025% to 15% by weight.

In one embodiment of the invention, in the compositions for treating acne, preferably emulsions, the biologically active compounds are present in concentrations ranging from 0.1% to 10% by weight and more preferably from 0.5% to 2% by weight relative to the total weight of the composition.

The micronized biologically active compound can be provided by various methods such as, for example, the air-jet method.

The particle size distribution of the biologically active compound is such that at least 80%, in numerical terms, and preferably at least 90%, in numerical terms, of the particles have a diameter ranging from 1 to 10 μm and at least 50%, in numerical terms, of the particles have a diameter of less than 5 μm. The mean particle diameter of the biologically active compound thus micronized is advantageously ranges from 3 to 5 μm. Preferably, at least 30% by number of the particles have a diameter ranging from 3 to 5 μm and even more preferably at least 50% by number of the particles have a diameter ranging from 3 to 5 μm.

The micronized biologically active compound is not solubilized in the compositions of the invention. By the expression “not solubilized” is intended a biologically active compound which is dissolved to less than 0.05% and preferably to less than 0.01% by weight relative to the weight of each of the other compounds, taken individually, of the composition.

The fatty phase of the emulsion according to the invention can comprise fatty substances conventionally employed in the application field envisaged. These are selected such that they do not solubilize the biologically active agent at a pH which is compatible with the skin.

Exemplary fatty substances are silicone fatty substances such as silicone oils, as well as non-silicone fatty substances such as plant, mineral, animal or synthetic oils.

Exemplary silicone fatty substances are poly(C₁-C₂₀)alkylsiloxanes and in particular those containing trimethylsilyl endgroups, preferably those whose viscosity is less than 0.06 m²/s, among which representative are linear polydimethylsiloxanes and alkylmethylpolysiloxanes such as cetyldimethicone (CTFA name); volatile silicone oils, such as cyclic volatile silicones containing from 3 to 8 and preferably from 4 to 5 silicon atoms, such as, for example, a cyclomethicone such as cyclotetradimethylsiloxane, cyclopentadimethylsiloxane or cyclohexadimethylsiloxane, cyclocopolymers such as dimethylsiloxane/methylalkylsiloxane, linear volatile silicones containing from 2 to 9 silicon atoms, such as, for example, hexamethyldisiloxane, hexyl heptamethyltrisiloxane or octyl heptamethyltrisiloxane; phenylsilicone oils.

And exemplary non-silicone fatty substances are the usual oils, such as isohexadecane, liquid paraffin, liquid petroleum jelly, perhydrosqualene, apricot oil, wheat germ oil, sweet almond oil, beauty-leaf oil, palm oil, castor oil, avocado oil, jojoba oil, olive oil or cereal germ oil; esters of fatty acids or of fatty alcohols, such as diisopropyl adipate, octyldodecyl myristate or (C₁₂-C₁₅)alkyl benzoates; acetyl glycerides; alkyl or polyalkyl octanoates, decanoates or ricinoleates; fatty acid triglycerides; glycerides; hydrogenated polyisobutene, hydrogenated oils that are solid at 25° C.; lanolins; fatty esters that are solid at 25° C. Other fatty substances which are exemplary are fatty acids such as stearic acid, fatty alcohols such as stearyl alcohol or cetyl alcohol or derivatives thereof, and waxes, or mixtures thereof.

These fatty substances can variously be selected by one skilled in this art in order to formulate a composition which has the desired properties, for example in terms of consistency or texture.

Thus, the fatty phase of the emulsion according to the invention advantageously constitutes from 5% to 50% by weight relative to the total weight of the composition and preferably from 12% to 25% by weight.

When the composition is for treating acne, the fatty substances are preferably selected from among dry to moderately dry oils, at contents preferably ranging from 5% to 30% by weight and more preferably from 12% to 25% by weight relative to the total weight of the composition.

By the expression “dry to moderately dry oil” is intended an oil which does not provide a sensation of greasiness on the skin and/or which does not leave a greasy film on the skin.

The dry to moderately dry oils are selected, for example, from among isohexadecane marketed under the trademark Arlamol HD by ICI, dioctylcyclohexane marketed under the trademark Cetiol S by Henkel, isopropyl palmitate marketed under the trademark Crodamol IPP by Croda, hydrogenated polyisobutene marketed under the trademark Polysynlane by NOF, diisopropyl adipate marketed under the trademark Ceraphyl 230 by ISP Van Dyk, dicaprylyl ether marketed under the trademark Cetiol OE by Henkel, isopropyl myristate marketed under the trademark Crodamol IPM by Croda, dipropylene glycol dipelargonate marketed under the trademark DPPG by Gattefosse, C₁₂₋₁₅ alkyl benzoate marketed under the trademark Finsolv TN by Finetex, cetostearyl isononanoate marketed under the trademark Cetiol SN by Henkel, cetostearyl ethylhexanoate marketed under the trademark Crodamol CAP by Croda, synthetic squalene marketed under the trademark Isolan RS by Goldschmidt, olive oil, octyl palmitate marketed under the trademark Crodamol OP by Croda, octyldodecyl myristate marketed under the trademark MODWL2949 by Gattefosse, caprylic/capric triglycerides marketed under the trademark Miglyol 812 by Hüls or marketed under the trademark Myritol 318 by Henkel.

Other dry to moderately dry oils can be used provided that they have sensory characteristics equivalent to those indicated above.

Thus, by way of example, other dry to moderately dry oils which can be formulated into the emulsions according to the invention are selected from among esters such as isopropyl palmitate, diesters such as diisopropyl adipate marketed by ISP Van Dyk under the trademark Ceraphyl 230, or marketed by Croda under the trademark Crodamol DA, ethers such as dicaprylyl ether and polyethers, hydrocarbons such as hydrogenated polyisobutene marketed under the trademark polysynlane by NOF or isohexadecane marketed by ICI under the trademark Arlamol HD, silicone oils such as cyclomethicones and dimethicones, or mixtures thereof.

When the biologically active compound is an agent or species which modulates differentiation and/or proliferation, such as, for example, an acidic retinoid such as 6-[7-(1-adamantyl)-6-methoxyethoxymethoxy-2-naphthyl]nicotinic acid, 6-[3-(1-adamantyl-4-hydroxyphenyl]-2-naphthoic acid, 6-(3,5,5,8,8-pentamethyl-5,6,7,8-tetrahydro-2-naphthylthio)nicotinic acid, 3-(3,5,5,8,8-pentamethyl-5,6,7,8-tetrahydro-2-naphthyl)phenylacrylic acid or 2-hydroxy-4-[7-(1-adamantyl)-6-methoxyethoxymethoxy-2-naphthyl]benzoic acid, the dry to moderately dry oils which can be formulated into the emulsions according to the invention are preferably selected from among the hydrocarbons such as hydrogenated polyisobutene, isohexadecane marketed by ICI under the trademark Arlamol HD, and silicone oils such as cyclomethicones and dimethicones, or mixtures thereof.

The aqueous phase of the emulsions according to the invention can comprise tap or distilled water, a floral water such as cornflower water, or a thermal spring water or natural mineral water selected, for example, from among eau de Vittel, waters from the Vichy basin, eau de Uriage, eau de la Roche Posay, eau de la Bourboule, eau d'Enghien-les-Bains, eau de Saint Gervais-les-Bains, eau de Néris-les-Bains, eau d'Allevard-les-Bains, eau de Digne, eau de Maizières, eau de Neyrac-les-Bains, eau de Lons-le-Saunier, Eaux Bonnes, eau de Rochefort, eau de Saint Christau, eau des Fumades, eau de Tercis-les-Bains, eau d'Avène or eau d'Aix-les-Bains.

Said aqueous phase advantageously constitutes from 30% to 95% by weight relative to the total weight of the composition, preferably from 60% to 80% by weight.

The pH of the compositions of this invention advantageously ranges from 5 to 7, preferably from 5 to 6. It will be adjusted to the desired value by addition of the usual inorganic or organic acids or bases.

The emulsions of the invention can also contain one or more wetting agents in concentrations preferably ranging from 0.1% to 10% and more preferably ranging from 2% to 2.5%. Exemplary of these wetting agents are compounds such as Poloxamers and more particularly Poloxamer 124 and/or Poloxamer 182, oxyethylenated sorbitol esters such as Polysorbates and more particularly Polysorbate 60 and/or Polysorbate 80.

The emulsions of the invention can also contain one or more pro-penetrating agents and/or wetting agents in concentrations preferably ranging from 1% to 20% and more preferably ranging from 2% to 6%. Exemplary of preferred pro-penetrating and/or wetting agents are compounds such as propylene glycol, glycerol and sorbitol.

The emulsions of the invention can also contain one or more gelling agents in concentrations preferably ranging from 0.05% to 5% and more preferably ranging from 0.1% to 1%. Exemplary of preferred gelling agents are compounds such as carboxyvinyl polymers (Carbomer), cellulose derivatives such as, for example, hydroxypropylmethylcellulose or hydroxyethylcellulose; xanthan gums, guar gums and the like, polyacrylamides such as the polyacrylamide/C13-14 isoparaffin/laureth-7 mixture, such as, for example, the product marketed by SEPPIC under the trademark Sepigel 305, or mixtures thereof.

The subject emulsions can also comprise any additive or adjuvant usually formulated into cosmetics or pharmaceuticals, such as sequestering agents, antioxidants, sunscreens, preservatives, fillers, dyes or colorants, fragrances, essential oils, cosmetic active agents, moisturizers, vitamins, essential fatty acids, sphingolipids, artificial tanning compounds such as DHA, and agents for soothing and protecting the skin such as allantoin. One skilled in this art will of course take care to select this or these optional additional compound(s), and/or the amounts thereof, such that the advantageous properties of the composition according to the invention are not, or are not substantially, adversely affected by the envisaged addition.

These additives and adjuvants can be present in the composition in a proportion of from 0% to 20% by weight relative to the total weight of the composition.

Exemplary sequestering agents include ethylenediaminetetraacetic acid (EDTA), as well as the derivatives or salts thereof, dihydroxyethylglycine, citric acid and tartaric acid, or mixtures thereof.

And exemplary preservatives include benzalkonium chloride, phenoxyethanol, benzyl alcohol, diazolidinylurea and parabens, or mixtures thereof.

In order to further illustrate the present invention and the advantages thereof, the following specific examples are given, it being understood that same are intended only as illustrative and in nowise limitative.

In said examples to follow, all parts and percentages are given by weight, unless otherwise indicated.

The following Examples 1-5 provide representative specific formulations according to the present invention.

### EXAMPLE 1

| | | |
|---|---|---|
| Phase A: | | |
| Glyceryl stearate and PEG-100 stearate | | 5.00% |
| Hydrogenated polyisobutene | | 11.00% |
| Propyl paraben | | 0.10% |
| Stearic acid | | 2.00% |
| Phase B: | | |
| Water | q.s. | 100% |
| Propylene glycol | | 2% |
| Disodium edetate | | 0.10% |
| Methyl paraben | | 0.10% |
| Phase C: | | |
| Nadifloxacin | | 1.00% |
| Poloxamer 124 | | 2.00% |
| Propylene glycol | | 2.00% |
| Copolymer of acrylic acid and | | 0.20% |
| alkyl methacrylate | | |
| Cyclomethicone | | 3.00% |
| 10% sodium hydroxide | q.s. | pH 5.5 |

Procedure:

The components of phase B were weighed and stirred with heating. The copolymer of acrylic acid and alkyl methacrylate was then incorporated.

Phase A was prepared separately by mixing and this phase A was heated on a water bath at 75° C.

Phase A was added to phase B, while maintaining the temperature at 75° C. with stirring. The mixture was then cooled and the cyclomethicone and the active phase were incorporated at 40° C. The pH was adjusted to 5.5 with sodium hydroxide.

A stable emulsion was obtained, at a pH that is compatible with the skin, and which is comfortable when applied, while at the same time avoiding a sticky effect or feel, i.e., a vehicle suited for the treatment of the indicated pathologies.

### EXAMPLE 2

| | | |
|---|---|---|
| Phase A: | | |
| Isohexadecane | | 5.00% |
| Hydrogenated polyisobutene | | 12.00% |
| Propyl paraben | | 0.10% |
| Sorbitan sesquiolate | | 0.15% |
| Ceteareth 20 | | 0.25% |
| Phase B: | | |
| Water | q.s. | 100% |
| Propylene glycol | | 2.00% |
| Disodium edetate | | 0.10% |
| Methyl paraben | | 0.10% |
| Phase C: | | |
| Nadifloxacin | | 1.00% |
| Poloxamer 124 | | 2.00% |
| Propylene glycol | | 2.00% |
| Copolymer of acrylic acid and | | 0.35% |
| alkyl methacrylate | | |
| Carbomer | | 0.10% |
| 10% sodium hydroxide | q.s. | pH 5.5 |

Procedure:

The procedure was essentially the procedure of Example 1.

A stable emulsion was obtained, at a pH that is compatible with the skin, and which is comfortable when applied, while at the same time avoiding a sticky effect or feel, i.e., a vehicle suitable for the treatment of the intended pathologies.

### EXAMPLE 3

| | | |
|---|---|---|
| Phase A: | | |
| Diisopropyl adipate | | 12.00% |
| Ceteareth 20 | | 0.25% |
| Phase B: | | |
| Water | q.s. | 100% |
| Propylene glycol | | 2.00% |
| Disodium edetate | | 0.10% |
| Benzalkonium chloride | | 0.05% |
| Phase C: | | |
| Nadifloxacin | | 1.00% |
| Poloxamer 124 | | 2.00% |
| Propylene glycol | | 2.00% |
| Copolymer of acrylic acid and | | 0.35% |
| alkyl methacrylate | | |
| Carbomer 980 | | 0.30% |
| Cyclomethicone | | 5% |
| 10% sodium hydroxide | q.s. | pH 5.5 |

Procedure:

The procedure was essentially the procedure of Example 1.

A stable emulsion was obtained, at a pH that is compatible with the skin, and which is comfortable when applied, while at the same time avoiding a sticky effect or feel, i.e., a vehicle suitable for the treatment of the intended pathologies.

### EXAMPLE 4

| | | |
|---|---|---|
| Phase A: | | |
| Diisopropyl adipate | | 15.00% |
| Ceteareth 20 | | 0.25% |
| PPG-15 stearyl ether marketed under | | 5% |
| the trademark Arlamol E | | |
| Propyl paraben | | 0.05% |
| Phase B: | | |
| Water | q.s. | 100% |
| Propylene glycol | | 3.00% |
| Disodium edetate | | 0.10% |
| Methyl paraben | | 0.10% |
| Phase C: | | |
| 3-(3,5,5,8,8-Pentamethyl-5,6,7,8-tetrahydro- | | 1.00% |
| 2-naphthyl)phenylacrylic acid | | |
| Poloxamer 182 | | 2.00% |
| Propylene glycol | | 2.00% |
| Copolymer of acrylic acid and | | |
| alkyl methacrylate | | 0.35% |
| Carbomer 980 | | 0.10% |
| Benzalkonium chloride | | 0.10% |
| 10% sodium hydroxide | q.s. | pH 5.5 |

Procedure:

The procedure was essentially the procedure of Example 1.

A stable emulsion was obtained, at a pH that is compatible with the skin, and which is comfortable when applied, while at the same time avoiding a sticky effect or feel, i.e., a vehicle suitable for the treatment of the intended pathologies.

### EXAMPLE 5

| | | |
|---|---|---|
| Phase A: | | |
| Isopropyl palmitate | | 12.00% |
| Ceteareth 20 | | 0.40% |
| Cyclomethicone | | 5% |
| Propyl paraben | | 0.10% |
| Phase B: | | |
| Purified water | q.s. | 100% |
| Propylene glycol | | 2.00% |
| Disodium edetate | | 0.10% |
| Copolymer of acrylic acid and | | 0.35% |
| alkyl methacrylate | | |
| Carbomer 980 | | 0.25% |
| Phenoxyethanol | | 1.00% |
| Phase C: | | |
| Nadifloxacin | | 1.00% |
| Poloxamer 124 | | 2.00% |
| Propylene glycol | | 2.00% |
| 10% sodium hydroxide | q.s. | pH 5.5 |

Procedure:

The procedure was essentially the procedure of Example 1.

A stable emulsion was obtained, at a pH that is compatible with the skin, and which is comfortable when applied, while at the same time avoiding a sticky effect or feel, i.e., a vehicle suitable for the treatment of the intended pathologies.

### EXAMPLE 6

This example reports stability studies of several formulations according to the present invention.

Various emulsions of the invention were tested as regards their chemical stability. The concentrations of biologically active compound set forth in the Table below were measured by HPLC:

Recovery: percentage of nadifloxacin existing in the product relative to the theoretical amount introduced.

| | | TABLE | | | |
|---|---|---|---|---|---|
| | | Recovery of nadifloxacin measured | | | |
| | | T0 | T1 month | T2 months | T3 months |
| Example 1 | T ambient | 99.6% | 100.0% | 100.6% | 100.9% |
| | T 45° C. | / | 101.4% | 100.7% | 101.8% |
| Example 2 | T ambient | 96.6% | 98.2% | 98.9% | 99.2% |
| | T 45° C. | / | 99.1% | 99.1% | 99.5% |
| Example 3 | T ambient | 95.9% | 97.7% | 98.2% | 97.8% |
| | T 45° C. | / | 98.4% | 99.8% | 99.8% |

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable cosmetic/pharmaceutical oil-in-water emulsion including a discontinuous fatty phase dispersed in a continuous aqueous phase and comprising an effective amount of at least one biologically active agent (A) and an effective amount of an emulsifying system (B) therefor, said at least one biologically active agent (A) being non-solubilized therein in micronized particulate state, at least 80%, numerically, of said micronized particles having diameters ranging from 1 to 10 μm and at least 50%, also numerically, having diameters of less than 5 μm.

2. The topically applicable cosmetic/pharmaceutical O/W emulsion as defined by claim 1, at least 90%, numerically, of said micronized particles having diameters ranging from 1 to 10 μm.

3. The topically applicable cosmetic/pharmaceutical O/W emulsion as defined by claim 1, said emulsifying system (B) comprising at least one copolymerizate of a major amount of a monoolefinically unsaturated C₃-C₆ carboxylic acid monomer, or anhydride thereof, with a minor amount of an acrylic acid fatty ester comonomer.

4. The topically applicable cosmetic/pharmaceutical O/W emulsion as defined by claim 3, said emulsifying system (B) comprising at least one copolymerizate of 80% to 98% by weight of said monoolefinically unsaturated C₃-C₆ carboxylic acid monomer, or anhydride thereof, with 20% to 2% by weight of an acrylic acid fatty ester comonomer.

5. The topically applicable cosmetic/pharmaceutical O/W emulsion as defined by claim 3, said carboxylic acid monomer having the formula: 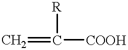
in which R is hydrogen, halogen, hydroxyl, a lactone group, a lactam group, a cyanogen (—C═N) group, a monovalent alkyl radical, an aryl radical, an alkylaryl radical, an aralkyl radical or a cycloaliphatic radical.

6. The topically applicable cosmetic/pharmaceutical O/W emulsion as defined by claim 5, said ester comonomer having the formula: 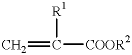
in which R¹ is hydrogen, methyl or ethyl and R² is a C₈-C₃₀ alkyl radical.

7. The topically applicable cosmetic/pharmaceutical O/W emulsion as defined by claim 6, said carboxylic acid monomer comprising acrylic acid, methacrylic acid, or mixtures thereof, and wherein said ester comonomer, R¹ is hydrogen or methyl and R² is a C₁₀-C₂₂ alkyl radical.

8. The topically applicable cosmetic/pharmaceutical O/W emulsion as defined by claim 3, said at least one copolymerizate comprising an acrylic/C₁₀-C₃₀-alkylacrylate copolymer.

9. The topically applicable cosmetic/pharmaceutical O/W emulsion as defined by claim 8, comprising from 0.05% to 2% by weight of said acrylic/C₁₀-C₃₀ alkylacrylate copolymer.

10. The topically applicable cosmetic/pharmaceutical O/W emulsion as defined by claim 3, comprising from 0.01% to 3% by weight of said at least one copolymerizate.

11. The topically applicable cosmetic/pharmaceutical O/W emulsion as defined by claim 1, further comprising at least one surfactant emulsifier.

12. The topically applicable cosmetic/pharmaceutical O/W emulsion as defined by claim 11, said at least one surfactant emulsifier comprising glyceryl or PEG-100 stearate, a polyoxyethylenated fatty acid ester, a polyoxyethylenated stearyl alcohol (2) combined with a polyethylenated stearyl alcohol (21), or mixture thereof.

13. The topically applicable cosmetic/pharmaceutical O/W emulsion as defined by claim 11, further comprising at least one co-surfactant.

14. The topically applicable cosmetic/pharmaceutical O/W emulsion as defined by claim 13, said at least one co-surfactant comprising a sorbitan ester, sorbitan sesquioleate, sorbitan isostearate, a fatty alkyl ether having a high HLB value, a fatty alkyl ether having a low HLB value, or mixture thereof.

15. The topically applicable cosmetic/pharmaceutical O/W emulsion as defined by claim 1, comprising up to 15% by weight of said emulsifying system (B).

16. The topically applicable cosmetic/pharmaceutical O/W emulsion as defined by claim 15, comprising from 0.05% to 8% by weight of said emulsifying system (B).

17. The topically applicable cosmetic/pharmaceutical O/W emulsion as defined by claim 16, comprising from 0.1% to 2% by weight of said emulsifying system (B).

18. The topically applicable cosmetic/pharmaceutical O/W emulsion as defined by claim 1, said at least one biologically active agent (A) being insoluble or difficultly soluble in water or a hydrophilic medium under pH conditions which are compatible with the skin.

19. The topically applicable cosmetic/pharmaceutical O/W emulsion as defined by claim 1, said at least one biologically active agent (A) comprising an agent which modulates skin differentiation and/or proliferation and/or pigmentation, an antibacterial agent, an antiparasitic agent, an antifungal agent, an antibiotic, a steroidal anti-inflammatory agent, a non-steroidal anti-inflammatory agent, an anaesthetic, an anti-pruriginous agent, an antiviral agent, a keratolytic agent, a free-radical scavenger, an antiseborrhoeic agent, an antidandruff agent, an anti-acne agent, an antimetabolite, an agent for combating hair loss and for promoting hair growth or vice versa, an antiseptic, or mixture thereof.

20. The topically applicable cosmetic/pharmaceutical O/W emulsion as defined by claim 19, comprising from 0.0001% to 20% by weight of said at least one biologically active agent (A).

21. The topically applicable cosmetic/pharmaceutical O/W emulsion as defined by claim 20, comprising from 0.025% to 15% by weight of said at least one biologically active agent (A).

22. The topically applicable cosmetic/pharmaceutical O/W emulsion as defined by claim 1, comprising from 5% to 50% by weight of said discontinuous fatty phase.

23. The topically applicable cosmetic/pharmaceutical O/W emulsion as defined by claim 22, comprising from 12% to 25% by weight of said at least one discontinuous fatty phase.

24. The topically applicable cosmetic/pharmaceutical O/W emulsion as defined by claim 1, said discontinuous fatty phase comprising a silicone fatty substance.

25. The topically applicable cosmetic/pharmaceutical O/W emulsion as defined by claim 24, said silicone fatty substance comprising a poly(C₁-C₂₀)alkylsiloxane or one containing trimethylsilyl endgroups, a linear polydimethylsiloxane, an alkylmethylpolysiloxane, cetyldimethicone, a volatile silicone oil, a cyclic volatile silicone having from 3 to 8 silicon atoms, a cyclomethicone, cyclotetradimethylsiloxane, cyclopentadimethylsiloxane, cyclohexadimethylsiloxane, a cyclocopolymer, a dimethylsiloxane/methylalkylsiloxane, a linear volatile silicone having from 2 to 9 silicon atoms, hexamethyldisiloxane, hexyl heptamethyltrisiloxane, octyl heptamethyltrisiloxane, a phenylsilicone oil, or mixture thereof.

26. The topically applicable cosmetic/pharmaceutical O/W emulsion as defined by claim 1, said discontinuous fatty phase comprising a non-silicone fatty oil, isohexadecane, a liquid paraffin, a liquid petroleum jelly, almond oil, perhydrosqualene, apricot oil, wheat germ oil, sweet almond oil, beauty-leaf oil, palm oil, castor oil, avocado oil, jojoba oil, olive oil, cereal germ oil, an ester of fatty acid or of a fatty alcohol, diisopropyl adipate, octyldodecyl myristate, a (C₁₂-C₁₅)alkyl benzoate, an acetyl glyceride, an alkyl or polyalkyl octanoate, a decanoate or ricinoleate, a fatty acid triglyceride, a glyceride, a hydrogenated polyisobutene, a hydrogenated oil that is solid at 25° C., a lanolin, a fatty ester that is solid at 25° C., or mixture thereof.

27. The topically applicable cosmetic/pharmaceutical O/W emulsion as defined by claim 1, said discontinuous fatty phase comprising a fatty acid or fatty alcohol, or derivative thereof, a wax, or mixture thereof.

28. The topically applicable cosmetic/pharmaceutical O/W emulsion as defined by claim 1, comprising from 30% to 95% by weight of said continuous aqueous phase.

29. The topically applicable cosmetic/pharmaceutical O/W emulsion as defined by claim 28, comprising from 60% to 80% by weight of said continuous aqueous phase.

30. The topically applicable cosmetic/pharmaceutical O/W emulsion as defined by claim 1, said continuous aqueous phase comprising a floral water, cornflower water, a thermal spring water, a natural mineral water, eau de Vittel, a water from the Vichy basin, eau de Uriage, eau de la Roche Posay, eau de la Bourboule, eau d'Enghien-les-Bains, eau de Saint Gervais-les-Bains, eau de Néris-les-Bains, eau d'Allevard-les-Bains, eau de Digne, eau de Maizières, eau de Neyrac-les-Bains, eau de Lons-le-Saunier, Eaux Bonnes, eau de Rochefort, eau de Saint Christau, eau des Fumades, eau de Tercis-les-Bains, eau d'Avène, eau d'Aix-les-Bains, or mixture thereof.

31. The topically applicable cosmetic/pharmaceutical O/W emulsion as defined by claim 1, further comprising at least one wetting agent.

32. The topically applicable cosmetic/pharmaceutical O/W emulsion as defined by claim 31, said at least one wetting agent comprising a poloxamer, an oxyethylenated sorbitol ester, a polysorbate, or mixture thereof.

33. The topically applicable cosmetic/pharmaceutical O/W emulsion as defined by claim 31, comprising from 01.% to 10% by weight of said at least one wetting agent.

34. The topically applicable cosmetic/pharmaceutical O/W emulsion as defined by claim 31, further comprising at least one pro-penetrating and/or wetting agent.

35. The topically applicable cosmetic/pharmaceutical O/W emulsion as defined by claim 34, said at least one pro-penetrating and/or wetting agent comprising propylene glycol, glycerol, sorbitol, or mixture thereof.

36. The topically applicable cosmetic/pharmaceutical O/W emulsion as defined by claim 34, comprising from 1% to 20% by weight of said at least one pro-penetrating and/or wetting agent.

37. The topically applicable cosmetic/pharmaceutical O/W emulsion as defined by claim 1, further comprising at least one gelling agent.

38. The topically applicable cosmetic/pharmaceutical O/W emulsion as defined by claim 37, said at least one gelling agent a carboxyvinyl polymer, a cellulose derivative, a xanthan gum, a guar gum, a polyacrylamide, or mixture thereof.

39. The topically applicable cosmetic/pharmaceutical O/W emulsion as defined by claim 37, comprising from 0.05% to 5% by weight of said at least one gelling agent.

40. The topically applicable cosmetic/pharmaceutical O/W emulsion as defined by claim 1, further comprising at least one sequestering agent, antioxidant, sunscreen, preservative, filler, dye or colorant, fragrance, essential oil, cosmetic active agent, moisturizer, vitamin, essential fatty acid, sphingolipid, artificial tanning agent, agent for soothing and protecting the skin, or mixture thereof.

41. A regime or regimen:
(1) for treating dermatological conditions/affliction associated with a keratinization disorder related to differentiation and proliferation, including common acne, comedones, polymorphonuclear leukocytes, rosacea, nodulocystic acne, acne conglobata, senile acne and secondary acne, including solar, medication-related or occupational acne, or
(2) for treating another keratinization disorder, including ichtyosis, an ichtyosiform state, Darier's disease, palmoplantar keratoderma, leukoplasia, a leukoplasiform state, and cutaneous or mucous (buccal) lichen, or
(3) for treating another dermatological condition/affliction associated with a keratinization disorder manifesting an inflammatory and/or immunoallergenic component, including all forms of psoriasis, whether cutaneous, mucous or ungual psoriasis, psoriatic rheumatism, cutaneous atopy, eczema, respiratory atopy, gingival hypertrophy, or for treating inflammatory conditions which manifest no keratinization disorder, including rosacea, or
(4) for treating all dermal or epidermal proliferations, whether benign or malignant and whether of viral or non-viral origin, including common warts, flat warts, verruciform epidermodysplasia, oral or florid papillomatoses and proliferations induced by ultraviolet radiation, basocellular or spinocellular epithelioma, or
(5) for treating bullosis or a collagen disease state, or
(6) for repairing or combating aging of the skin, whether photoinduced or chronological aging, or for reducing pigmentations and actinic keratosis, or any pathology associated with chronological or actinic aging, or
(7) for preventing or curing the stigmata of epidermal and/or dermal atrophy induced by local or systemic corticosteroids, or other form of cutaneous atrophy, or
(8) for the preventive or curative treatment of cicatrization disorders, for preventing or repairing stretch marks, or for promoting cicatrization, or
(9) for combating disorders of sebaceous functioning, including acneic hyperseborrhoea or simple seborrhoea, or
(10) for the preventive or curative treatment of cancerous or precancerous disease states, or
(11) for the treatment of inflammatory conditions/afflictions, including arthritis, or
(12) for the treatment of any skin condition/affliction of viral origin, or
(13) for the preventive or curative treatment of alopecia, or
(14) for the treatment of dermatological conditions/afflictions manifesting an immunological component, or
(15) for the treatment of skin disorders caused by exposure to UV radiation, or
(16) for the treatment of dermatological conditions/afflictions associated with inflammation or infection of the tissues surrounding the hair follicles, including colonization or microbial infection, impetigo, seborrhoeic dermatitis, folliculitis, sycosis barbae, or
(17) for accelerating or promoting hair loss, comprising topically applying onto the skin, scalp and/or hair of a candidate subject in need of such treatment, for such period of time as required to elicit the desired biological response, a topically applicable cosmetic/pharmaceutical oil-in-water emulsion including a discontinuous fatty phase dispersed in a continuous aqueous phase and comprising an effective amount of at least one thus-biologically active agent (A) and an effective amount of an emulsifying system (B) therefor, said at least one biologically active agent (A) being non-solubilized therein in micronized particulate state, at least 80%, numerically, of said micronized particles having diameters ranging from 1 to 10 μm and at least 50%, also numerically, having diameters of less than 5 μm.

42. The regime/regimen as defined by claim 41, comprising preventively or curatively treating acne.

43. The regime/regimen as defined by claim 1, said at least one biologically active agent (A) comprising an agent which modulates proliferation and/or differentiation, an antibiotic, an antibacterial agent, an antifungal agent, an antiparasitic agent, or mixture thereof.

44. The regime/regimen as defined by claim 1, said at least one biologically active agent (A) comprising nadifloxacin, 6-[7-(1-adamantyl)-6-methoxyethoxymethoxy-2-naphthyl]nicotinic acid, 6-[3-(1-adamantyl-4-hydroxyphenyl]-2-naphthoic acid, 6-(3,5,5,8,8-pentamethyl-5,6,7,8-tetrahydro-2-naphthylthio)nicotinic acid, 3-(3,5,5,8,8-pentamethyl-5,6,7,8-tetrahydro-2-naphthyl)phenylacrylic acid, 2-hydroxy-4-[7-(1-adamantyl)-6-methoxyethoxymethoxy-2-naphthyl]benzoic acid, or mixture thereof.

45. The regime/regimen as defined by claim 1, said discontinuous fatty phase comprising at least one dry to moderately dry fatty oil.

46. The regime/regimen as defined by claim 45, said at least one dry to moderately dry fatty oil comprising isohexadecane, dioctylcyclohexane, isopropyl palmitate, hydrogenated polyisobutene, diisopropyl adipate, dicaprylyl ether, isopropyl myristate, dipropylene glycol dipelargonate, a C₁₂-C₁₅ alkyl benzoate, cetostearyl isononanoate, cetostearyl ethylhexanoate, synthetic squalene, olive oil, octyl palmitate, octyldodecyl myristate, a caprylic/capric triglycerides, or mixture thereof.

47. The topically applicable cosmetic/pharmaceutical O/W emulsion as defined by claim 1, having a pH ranging from 5 to 7.

48. The topically applicable cosmetic/pharmaceutical O/W emulsion as defined by claim 47, having a pH ranging from 5 to 6.

49. A regime or regiment for therapeutically treating inflammation or infection of the skin tissue surrounding hair follicles, comprising topically applying onto this skin tissue of a candidate subject in need of such treatment, for such period of time as required to elicit the desired biological response, a topically applicable cosmetic/pharmaceutical oil-in-water emulsion including a discontinuous fatty phase dispersed in a continuous aqueous phase and comprising an effective amount of at least one thus-biologically active agent (A) and an effective amount of an emulsifying system (B) therefor, said at least one biologically active agent (A) being non-solubilized therein in micronized particulate state, at least 80%, numerically, of said micronized particles having diameters ranging from 1 to 10 μm and at least 50%, also numerically, having diameters of less than 5 μm.
